(19) United States
(12) Patent Application Publication
(10) Pub. No.: US 2006/0277631 A1
(43) Pub. Date: Dec. 7, 2006

(54) Genes upregulated in a tomato plant having an increased anthocyanin content phenotype

(75) Inventors: Stephanie K Clendennen, Kingsport, TN (US); Jonathan Lightner, Des Moines, IA (US)

Correspondence Address: KLARQUIST SPARKMAN, LLP, 121 S.W. SALMON STREET, SUITE 1600, PORTLAND, OR, 97204, US

(73) Assignee: EXELIXIS PLANT SCIENCES, INC., Portland, OR (US)

(21) Appl. No.: 10/554,386

(22) PCT Filed: Apr. 26, 2004
(86) PCT No.: PCT/US04/12826

§ 371 Date: Aug. 8, 2006

Related U.S. Application Data
(60) Provisional application No. 60/465,605, filed on Apr. 25, 2003.

(51) Int. Cl. A01H 1/00; C12N 15/82; C07H 15/02; C07D 311/02

(52) U.S. Cl. 800/282; 549/403; 536/8

(57) ABSTRACT

Tomato anthocyanin vacuolar transporter (“MTP77”) and chalcone isomerase (“MTP96”) are up-regulated in tomato plants that overexpress the ANT1 gene. Plant transformation vectors comprising isolated MTP77 or MTP96 polynucleotides can be made to generate transgenic plants having increased anthocyanin content relative to control plants.

### REFERENCE TO RELATED APPLICATIONS

This application claims priority to U.S. provisional patent application 60/465,605 filed Apr. 25, 2003, the contents of which are hereby incorporated by reference.

### FIELD OF THE INVENTION

The present invention relates to genes and methods for altering anthocyanin content in plants.

### BACKGROUND OF THE INVENTION

Anthocyanins have been associated with many important physiological and developmental functions in the plants, including, modification of the quantity and quality of captured light (Barker D H et al, Plant Cell and Environment 20: 617-624, 1977.); protection from the effects of UV-B radiation (Burger J and Edwards G E. Plant and Cell Physiology 37: 395-399, 1996; Klaper R et al., Photochemistry and Photobiology 63: 811-813, 1996); defense against herbivores (Coley and Kusar. In: Mulkey S S, Chazdon R L, Smith A P, eds. Tropical Forest Plant Ecophysiology. New York: Chapman and Hall 305-335, 1996); and protection from photoinhibition (Gould K S, et al., Nature 378: 241-242, 1995; and Dodd I C et al,. Jounal of Experimental Botany 49: 1437-1445, 1998); and scavenging of reactive oxygen intermediates in stressful environments (Furuta S et al., Sweetpotato Res Front (KNAES, Japan) 1:3, 1995; Sherwin H W and Farrant J M., Plant Growth Regulation 24: 203-210, 1998; and Yamasaki H Trends in Plant Science 2:7-8, 1997).

Anthocyanins have demonstrated anti-oxidant activity, suggesting a role in protecting against cancer, cardiovascular and liver diseases (Kamei H et al., J Clin Exp Med 164: 829, 1993; Suda I, et al., 1997. Sweetpotato Res Front (KNAES, Japan) 4:3, 1997; and Wang C J, et al., H Food Chem Toxicology 38: 411-416, 2000). Thus, anthocyanin-rich foods and extracts have been studied for their utility in a variety of therapeutic applications (e.g. Katsube et al., J Agric Food Chem (2003) 51(1):68-75; Renaud et al., Lancet (1992) 339:1523-1526; and Natella et al., J Agric Food Chem (2002) 50(26):7720-7725). There is also interest in the use of anthocyanin-rich plant species in the production of natural dyes (Venturi and Piccaglia, “The Rediscovery of Dye Plants as Promising “Non Food Crops””. Interactive European Network for Industrial Crops and their Applications, Newsletter no. 10, Nov. 1999).

Most of the structural genes that encode the enzymes required for anthocyanin biosynthesis and modification have been isolated, and analysis of mutants in Antirrhinum, Arabidopsis, maize, and petunia, has led to further identification of a number of genes involved in regulating the expression of the structural anthocyanin genes (Mol et al., Trends Plant Sci (1998) 3:2122-217; Winkel-Shirley, Plant Physiol (2001) 126:485-493). Many steps in anthocyanin biosynthesis are shared among plant species, while the regulatory elements that underlie the expression level and pattern of genes encoding these enzymes are diverse. In Petunia, AN2 encodes a MYB domain protein that is orthologous to C1 from maize (Quattrocchio P et al., 1999, Plant Cell 11:1433-1444), and Arabidopsis genes PAP1 and PAP2 (Borevitz et al., Plant Cell. December 2000;12(12):2383-2394). The Anthocyanin1 gene (AN1) of petunia encodes a basic helix-loop-helix (bHLH) protein that activates the transcription of the structural anthocyanin gene Dihdroflavonol Reductates (DFR). The expression of AN1 is regulated by AN2 (Spelt et al., Plant Cell. September 2000;12(9):1619-32). In Arabidopsis, two other transcription factors have been implicated in controlling the accumulation of flavonoids: the homeodomain protein Anthocyaninless2 (ANL2) is required for anthocyanin accumulation in subepidermal cells, while and the zinc finger protein, TT1, is involved in the accumulation of proanthocyanidin polymers in the seed coat (Kubo et al., Plant Cell. July 1999;11(7):1217-26.; Sagasser et al., Genes Dev. Jan. 1, 2002; 16(1):138-49). The tomato ANT1 gene encodes a Myb-related transcription factor that when overexpressed results in modified anthocyanin content that results in a purple coloration in the leaves and fruit having a deeper red color (WO 02/055658).

### SUMMARY OF THE INVENTION

The invention is directed to tomato anthocyanin vacuolar transporter (designated “MTP77”) and chalcone isomerase (designated “MTP96”), which are up-regulated in tomato plants that overexpress the ANT1 gene. In one aspect, the invention provides an isolated polynucleotide comprising a nucleic acid sequence which encodes or is complementary to a sequence which encodes MTP96 or MTP77, or orthologs or variants thereof having at least 80% sequence identity to the amino acid sequence presented as SEQ ID NO:2 or SEQ ID NO:4. Plant transformation vectors comprising the isolated polynucleotides may be made to generate transgenic plants having increased anthocyanin content relative to control plants.

### DETAILED DESCRIPTION OF TIE INVENTION

Definitions

Unless otherwise indicated, all technical and scientific terms used herein have the same meaning as they would to one skilled in the art of the present invention. Practitioners are particularly directed to Sambrook et al. Molecular Cloning: A Laboratory Manual (Second Edition), Cold Spring Harbor Press, Plainview, N.Y.,1989; and Ausubel F M et al. Current Protocols in Molecular Biology, John Wiley & Sons, New York, N.Y., 1993, for definitions and terms of the art.

All publications cited herein are expressly incorporated herein by reference for the purpose of describing and disclosing compositions and methodologies that might be used in connection with the invention. All cited patents, patent publications, and sequence and other information in referenced websites are also incorporated by reference.

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

A “heterologous” nucleic acid construct or sequence has a portion of the sequence which is not native to the plant cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous“nucleic acid construct may contain a control sequence/DNA coding sequence combination that is the same as, or different from a control sequence/DNA coding sequence combination found in the native plant.

As used herein, the term “gene” means the segment of DNA involved in producing a polypeptide chain, which may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, “percent (%) sequence identity” with respect to a subject sequence, or a specified portion of a subject sequence, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1990) 215:403-410; blast.wustl.edulblast/README.html website) with all the search parameters set to default values. The HSP S and HSP S2 parameters are dynamic values and are established by the program itself depending upon the composition of the particular sequence and composition of the particular database against which the sequence of interest is being searched. A % identity value is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation.

The term “% homology” is used interchangeably herein with the term “% identity.” A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, for example, Sambrook, et al, supra, Chapters 9 and 11, and in Ausubel, F. M., et al, supra). An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

As used herein, the terms “transformed”, “stably transformed” or “transgenic” with reference to a plant cell means the plant cell has a non-native (heterologous) nucleic acid sequence integrated into its genome which is maintained through two or more generations.

As used herein, the term “expression” refers to the process by which a polypeptide is produced based on the nucleic acid sequence of a gene. The process includes both transcription and translation.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

As used herein, a “plant cell” refers to any cell derived from a plant, including cells from undifferentiated tissue (e.g., callus) as well as plant seeds, pollen, progagules and embryos.

As used herein, the terms “native” and “wild-type” relative to a given plant trait or phenotype refers to the form in which that trait or phenotype is found in the same variety of plant in nature.

As used herein, the term “modified” regarding a plant trait, refers to a change in the phenotype of a transgenic plant relative to a non-transgenic plant, as it is found in nature.

As used herein, the term “T₁” refers to the generation of plants from the seed of T₀ plants. The T₁ generation is the first set of transformed plants that can be selected by application of a selection agent, e.g., an antibiotic or herbicide, for which the transgenic plant contains the corresponding resistance gene.

As used herein, the term “T₂” refers to the generation of plants by self-fertilization of the flowers of T₁ plants, previously selected as being transgenic.

As used herein, the term “plant part” includes any plant organ or tissue including without limitation, seeds, embryos, meristematic regions, callus tissue, leaves, roots, shoots, gametophytes, sporophytes, pollen, and microspores. Plant cells can be obtained from any plant organ or tissue and cultures prepared therefrom. The class of plants which can be used in the methods of the present invention is generally as broad as the class of higher plants amenable to transformation techniques, including both monocotyledenous and dicotyledenous plants.

As used herein, “transgenic plant” includes reference to a plant that comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic.

Thus a plant having within its cells a heterologous polynucleotide is referred to herein as a “transgenic plant”. The heterologous polynucleotide can be either stably integrated into the genome, or can be extra-chromosomal. Preferably, the polynucleotide of the present invention is stably integrated into the genome such that the polynucleotide is passed on to successive generations. The polynucleotide is integrated into the genome alone or as part of a recombinant expression cassette. “Transgenic” is used herein to include any cell, cell line, callus, tissue, plant part or plant, the genotype of which has been altered by the presence of heterologous nucleic acids including those transgenics initially so altered as well as those created by sexual crosses or asexual reproduction of the initial transgenics.

A plant cell, tissue, organ, or plant into which the recombinant DNA constructs containing the expression constructs have been introduced is considered “transformed”, “transfected”, or “transgenic”. A transgenic or transformed cell or plant also includes progeny of the cell or plant and progeny produced from a breeding program employing such a transgenic plant as a parent in a cross and exhibiting an altered phenotype resulting from the presence of a recombinant nucleic acid sequence. Hence, a plant of the invention will include any plant which has a cell containing a construct with introduced nucleic acid sequences, regardless of whether the sequence was introduced into the directly through transformation means or introduced by generational transfer from a progenitor cell which originally received the construct by direct transformation.

The terms “Anthocyanin 1” and “ANT1”, as used herein encompass native Anthocyanin 1 (ANT1) nucleic acid and amino acid sequences, homologues, variants and fragments thereof.

The term “MTP” is used to refer to genes and their encoded proteins that are up-regulated in tomato plants that overexpress ANT. Specifically, MTP77 is used to refer to a tomato anthocyanin permease nucleic acid molecule of SEQ ID NO:1 or, depending on the context used, the protein encoded thereby having the amino acid sequence of SEQ ID NO:2. MTP96 is used to refer to a tomato chalcone isomerase nucleic acid molecule of SEQ ID NO:3 or the protein encoded thereby having the amino acid sequence of SEQ ID NO:4

An “isolated” MTP nucleic acid molecule or protein is an MTP nucleic acid molecule or protein that is identified and separated from at least one contaminant nucleic acid molecule or protein with which it is ordinarily associated in the natural source of the MTP nucleic acid or protein. An isolated MTP nucleic acid molecule or protein is other than in the form or setting in which it is found in nature. However, an isolated MTP nucleic acid molecule includes MTP nucleic acid molecules contained in cells that ordinarily express MTP where, for example, the nucleic acid molecule is in a chromosomal location different from that of natural cells.

As used herein, the term “mutant” with reference to a polynucleotide sequence or gene differs from the corresponding wild type polynucleotide sequence or gene either in terms of sequence or expression, where the difference contributes to a modified plant phenotype or trait. Relative to a plant or plant line, the term “mutant” refers to a plant or plant line which has a modified plant phenotype or trait, where the modified phenotype or trait is associated with the modified expression of a wild type polynucleotide sequence or gene.

Generally, a “variant” polynucleotide sequence encodes a “variant” amino acid sequence which is altered by one or more amino acids from the reference polypeptide sequence. The variant polynucleotide sequence may encode a variant amino acid sequence having “conservative” or “non-conservative” substitutions. Variant polynucleotides may also encode variant amino acid sequences having amino acid insertions or deletions, or both.

As used herein, the term “phenotype” may be used interchangeably with the term “trait”. The terms refer to a plant characteristic that is readily observable or measurable and results from the interaction of the genetic make-up of the plant with the environment in which it develops. Such a phenotype includes chemical changes in the plant make-up resulting from enhanced gene expression which may or may not result in morphological changes in the plant, but which are measurable using analytical techniques known to those of skill in the art.

MTP Nucleic Acids

The invention is directed to tomato anthocyanin vacuolar transporter (designated “MTP77”) and chalcone isomerase (designated “MTP96”), which, as detailed in the examples below, were found to be up-regulated in tomato plants that overexpress the ANT1 gene and that have an increased anthocyanin content phenotype.

An MTP gene may be used in the development of transgenic plants having a desired phenotype. This may be accomplished using the native MTP sequence, a variant MTP sequence or a homologue or fragment thereof.

An MTP nucleic acid sequence of this invention may be a DNA or RNA sequence, derived from genomic DNA, cDNA or mRNA. The nucleic acid sequence may be cloned, for example, by isolating genomic DNA from an appropriate source, and amplifying and cloning the sequence of interest using PCR. Alternatively, nucleic acid sequence may be synthesized, either completely or in part, especially where it is desirable to provide plant-preferred sequences. Thus, all or a portion of the desired structural gene (that portion of the gene which encodes a polypeptide or protein) may be synthesized using codons preferred by a selected host.

The invention provides a polynucleotide comprising a nucleic acid sequence which encodes or is complementary to a sequence which encodes an MTP polypeptide having the amino acid sequence presented in SEQ ID NO:2 or SEQ ID NO:4 and a polynucleotide sequence identical over its entire length to the MTP nucleic acid sequence presented SEQ ID NO:1 or SEQ ID NO:3. The invention also provides the coding sequence for the mature MTP polypeptide, a variant or fragment thereof, as well as the coding sequence for the mature polypeptide or a fragment thereof in a reading frame with other coding sequences, such as those encoding a leader or secretory sequence, a pre-, pro-, or prepro-protein sequence.

An MTP polynucleotide can also include non-coding sequences, including for example, but not limited to, non-coding 5′ and 3′ sequences, such as the transcribed, untranslated sequences, termination signals, ribosome binding sites, sequences that stabilize mRNA, introns, polyadenylation signals, and additional coding sequence that encodes additional amino acids. For example, a marker sequence can be included to facilitate the purification of the fused polypeptide. Polynucleotides of the present invention also include polynucleotides comprising a structural gene and the naturally associated sequences that control gene expression.

When an isolated polynucleotide of the invention comprises an MTP nucleic acid sequence flanked by non-MTP nucleic acid sequence, the total length of the combined polynucleotide is typically less than 25 kb, and usually less than 20 kb, or 15 kb, and in some cases less than 10 kb, or 5 kb.

In addition to the MTP nucleic acid and corresponding polypeptide sequences described herein, MTP variants can be prepared by introducing appropriate nucleotide changes into the MTP nucleic acid sequence; by synthesis of the desired MTP polypeptide or by altering the expression level of the MTP gene in plants. For example, amino acid changes may alter post-translational processing of the MTP polypeptide, such as changing the number or position of glycosylation sites or altering the membrane anchoring characteristics.

In one aspect, preferred MTP coding sequences include a polynucleotide comprising a nucleic acid sequence which encodes or is complementary to a sequence which encodes an MTP polypeptide having at least 50%, 60%, 70%, 75%, 80%, 85%, 90%, 95% or more sequence identity to the amino acid sequence presented in SEQ ID NO:2 or 4.

In another aspect, preferred variants include an MTP polynucleotide sequence that is at least 50% to 60% identical over its entire length to the MTP nucleic acid sequence presented as SEQ ID NO:1 or 3, and nucleic acid sequences that are complementary to such an MTP sequence. More preferable are MTP polynucleotide sequences comprise a region having at least 70%, 80%, 85%, 90% or 95% or more sequence identity to the MTP sequence presented as SEQ ID NO: 1 or 3.

In a related aspect, preferred variants include polynucleotides that are be “selectively hybridizable” to the MTP polynucleotide sequence presented as SEQ ID NO:1 or 3.

Sequence variants also include nucleic acid molecules that encode the same polypeptide as encoded by the MTP polynucleotide sequence described herein. Thus, where the coding frame of an identified nucleic acid molecule is known, for example by homology to known genes or by extension of the sequence, a number of coding sequences can be produced as a result of the degeneracy of the genetic code. For example, the triplet CGT encodes the amino acid arginine. Arginine is alternatively encoded by CGA, CGC, CGG, AGA, and AGG. Such substitutions in the coding region fall within the sequence variants that are covered by the present invention. Any and all of these sequence variants can be utilized in the same way as described herein for the identified MTP parent sequence, SEQ ID NO:1 or3.

Such sequence variants may or may not selectively hybridize to the parent sequence. This would be possible, for example, when the sequence variant includes a different codon for each of the amino acids encoded by the parent nucleotide. In accordance with the present invention, also encompassed are sequences that are at least 70% identical to such degeneracy-derived sequence variants.

Although MTP nucleotide sequence variants are preferably capable of hybridizing to the nucleotide sequences recited herein under conditions of moderately high or high stringency, there are, in some situations, advantages to using variants based on the degeneracy of the code, as described above. For example, codons may be selected to increase the rate at which expression of the peptide occurs in a particular prokaryotic or eukaryotic organism, in accordance with the optimum codon usage dictated by the particular host organism. Alternatively, it may be desirable to produce RNA having longer half lives than the mRNA produced by the recited sequences.

Variations in the native full-length MTP nucleic acid sequence described herein, may be made, for example, using any of the techniques and guidelines for conservative and non-conservative mutations, as generally known in the art, oligonucleotide-mediated (site-directed) mutagenesis, alanine scanning, and PCR mutagenesis. Site-directed mutagenesis (Kunkel T A et al., Methods Enzynol. 204:125-39, 1991); cassette mutagenesis (Crameri A and Stemmer W P, Bio Techniques 18(2):194-6, 1995.); restriction selection mutagenesis (Haught C et al. BioTechniques 16(1):47-48, 1994), or other known techniques can be performed on the cloned DNA to produce nucleic acid sequences encoding MTP variants.

In addition, the gene sequences may be synthesized, either completely or in part, especially where it is desirable to provide host-preferred sequences. Thus, all or a portion of the desired structural gene (that portion of the gene which encodes the protein) may be synthesized using codons preferred by a selected host. Host-preferred codons may be determined, for example, from the codons used most frequently in the proteins expressed in a desired host species.

It is preferred that an MTP polynucleotide encodes an MTP polypeptide that retains substantially the same biological function or activity as the mature MTP polypeptide encoded by the polynucleotide set forth as SEQ ID NO: 1 or 3.

Variants also include fragments of the MTP polynucleotide of the invention, which can be used to synthesize a full-length MTP polynucleotide. Preferred embodiments include polynucleotides encoding polypeptide variants wherein 5 to 10, 1 to 5, 1 to 3, 2, 1 or no amino acid residues of an MTP polypeptide sequence of the invention are substituted, added or deleted,′ in any combination. Particularly preferred are substitutions, additions, and deletions that are silent such that they do not alter the properties or activities of the polynucleotide or polypeptide.

A nucleotide sequence encoding an MTP polypeptide can also be used to construct hybridization probes for further genetic analysis. Screening of a cDNA or genomic library with the selected probe may be conducted using standard procedures, such as described in Sambrook et al., supra). Hybridization conditions, including moderate stringency and high stringency, are provided in Sambrook et al., supra.

The probes or portions thereof may also be employed in PCR techniques to generate a pool of sequences for identification of closely related MTP sequences. When MTP sequences are intended for use as probes, a particular portion of an MTP encoding sequence, for example a highly conserved portion of the coding sequence may be used.

For example, an MTP nucleotide sequence may be used as a hybridization probe for a cDNA library to isolate genes, for example, those encoding naturally-occurring variants of MTP from other plant species, which have a desired level of sequence identity to the MTP nucleotide sequence disclosed in SEQ ID NO: 1 or 3. Exemplary probes have a length of about 20 to about 50 bases.

In another exemplary approach, a nucleic acid encoding an MTP polypeptide may be obtained by screening selected cDNA or genomic libraries using the deduced amino acid sequence disclosed herein, and, if necessary, using conventional primer extension procedures as described in Sambrook et al., supra, to detect MTP precursors and processing intermediates of mRNA that may not have been reverse-transcribed into cDNA.

As discussed above, nucleic acid sequences of this invention may include genomic, cDNA or mRNA sequence. By “encoding” is meant that the sequence corresponds to a particular amino acid sequence either in a sense or anti-sense orientation. By “extrachromosomal” is meant that the sequence is outside of the plant genome of which it is naturally associated. By “recombinant” is meant that the sequence contains a genetically engineered modification through manipulation via mutagenesis, restriction enzymes, and the like.

Once the desired form of an MTP nucleic acid sequence, homologue, variant or fragment thereof, is obtained, it may be modified in a variety of ways. Where the sequence involves non-coding flanking regions, the flanking regions may be subjected to resection, mutagenesis, etc. Thus, transitions, transversions, deletions, and insertions may be performed on the naturally occurring sequence.

With or without such modification, the desired form of the MTP nucleic acid sequence, homologue, variant or fragment thereof, may be incorporated into a plant expression vector for transformation of plant cells.

MTP Polypeptides

In one preferred embodiment, the invention provides an MTP polypeptide, having a native mature or full-length MTP polypeptide sequence comprising the sequence presented in SEQ ID NO:2 or 4. An MTP polypeptide of the invention can be the mature MTP polypeptide, part of a fusion protein or a fragment or variant of the MTP polypeptide sequence presented in SEQ ID NO:2 or 4.

Ordinarily, an MTP polypeptide of the invention has at least 50% to 60% identity to an MTP amino acid sequence over its entire length. More preferable are MTP polypeptide sequences that comprise a region having at least 70%, 80%, 85%, 90% or 95% or more sequence identity to the MTP polypeptide sequence of SEQ ID NO:2 or 4.

Fragments and variants of the MTP polypeptide sequence of SEQ ID NO:2 or 4, are also considered to be a part of the invention. A fragment is a variant polypeptide that has an amino acid sequence that is entirely the same as part but not all of the amino acid. sequence of the previously described polypeptides. Exemplary fragments comprises at least 10, 20, 30, 40, 50, 75, or 100 contiguous amino acids of SEQ ID NO:2 or 4. The fragments can be “free-standing” or comprised within a larger polypeptide of which the fragment forms a part or a region, most preferably as a single continuous region. Preferred fragments are biologically active fragments, which are those fragments that mediate activities of the polypeptides of the invention, including those with similar activity or improved activity or with a decreased activity. Also included are those fragments that antigenic or immunogenic in an animal, particularly a human.

MTP polypeptides of the invention also include polypeptides that vary from the MTP polypeptide sequence of SEQ ID NO:2 or 4. These variants may be substitutional, insertional or deletional variants. The variants typically exhibit the same qualitative biological activity as the naturally occurring analogue, although variants can also be selected which have modified characteristics as further described below.

A “substitution” results from the replacement of one or more nucleotides or amino acids by different nucleotides or amino acids, respectively.

An “insertion” or “addition” is that change in a nucleotide or amino acid sequence which has resulted in the addition of one or more nucleotides or amino acid residues, respectively, as compared to the naturally occurring sequence.

A “deletion” is defined as a change in either nucleotide or amino acid sequence in which one or more nucleotides or amino acid residues, respectively, are absent.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances.

Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids.

Substitutions are generally made in accordance with known “conservative substitutions”. A “conservative substitution” refers to the substitution of an amino acid in one class by an amino acid in the same class, where a class is defined by common physicochemical amino acid side chain properties and high substitution frequencies in homologous proteins found in nature (as determined, e.g., by a standard Dayhoff frequency exchange matrix or BLOSUM matrix). (See generally, Doolittle, R. F., OF URFS and ORFS (University Science Books, Calif., 1986.))

A “non-conservative substitution” refers to the substitution of an amino acid in one class with an amino acid from another class.

MTP polypeptide variants typically exhibit the same qualitative biological activity as the naturally occurring analogue, although variants also are selected to modify the characteristics of the MTP polypeptide, as needed. For example, glycosylation sites, and more particularly one or more O-linked or N-linked glycosylation sites may be altered or removed. For example, amino acid changes may alter post-translational processes of the MTP polypeptide, such as changing the number or position of glycosylation sites or altering the membrane anchoring characteristics.

The variations can be made using methods known in the art such as oligonucleotide-mediated (site-directed) mutagenesis, alanine scanning, and PCR mutagenesis. Site-directed mutagenesis (Carter et al., Nucl. Acids Res. 13:4331, 1986; Zoller et al., Nucl. Acids Res. 10:6487, 1987), cassette mutagenesis (Wells et al., Gene 34:315, 1985), restriction selection mutagenesis (Wells et al., Philos. Trans. R. Soc. London SerA 317:415, 1986) or other known techniques can be performed on the cloned DNA to produce the MTP polypeptide-encoding variant DNA.

Also included within the definition of MTP polypeptides are other related MTP polypeptides. Thus, probe or degenerate PCR primer sequences may be used to find other related polypeptides. Useful probe or primer sequences may be designed to all or part of the MTP polypeptide sequence, or to sequences outside the coding region. As is generally known in the art, preferred PCR primers are from about 15 to about 35 nucleotides in length, with from about 20 to about 30 being preferred, and may contain inosine as needed. The conditions for the PCR reaction are generally known in the art.

Covalent modifications of MTP polypeptides are also included within the scope of this invention. For example, the invention provides MTP polypeptides that are a mature protein and may comprise additional amino or carboxyl-terminal amino acids, or amino acids within the mature polypeptide (for example, when the mature form of the protein has more than one polypeptide chain). Such sequences can, for example, play a role in the processing of a protein from a precursor to a mature form, allow protein transport, shorten or lengthen protein half-life, or facilitate manipulation of the protein in assays or production. Cellular enzymes can be used to remove any additional amino acids from the mature protein (Creighton, T. E., PROTEINS: STRUCTURE AND MOLECULAR PROPERTIES, W. H. Freeman & Co., San Francisco, pp. 79-86, 1983).

In a preferred embodiment, overexpression of an MTP polypeptide or variant thereof is associated with the previously described ANT1 phenotype (WO 02/055658).

MTP Orthologs

The methods of the invention may use orthologs of the MTP. Methods of identifying the orthologs in other plant species are known in the art. Normally, orthologs in different species retain the same function, due to presence of one or more protein motifs and/or 3-dimensional structures. In evolution, when a gene duplication event follows speciation, a single gene in one species, such as Arabidopsis, may correspond to multiple genes (paralogs) in another. As used herein, the term “orthologs” encompasses paralogs. When sequence data is available for a particular plant species, orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210).

Programs for multiple sequence alignment, such as CLUSTAL (Thompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. Nucleic acid hybridization methods may also be used to find orthologous genes and are preferred when sequence data are not available. Degenerate PCR and screening of cDNA or genomic DNA libraries are common methods for finding related gene sequences and are well known in the art (see, e.g., Sambrook, 1989). For instance, methods for generating a cDNA library from the plant species of interest and probing the library with partially homologous gene probes are described in Sambrook et al. A highly conserved portion of the MTP coding sequence may be used as a probe. MTP ortholog nucleic acids may hybridize to the nucleic acid of SEQ ID NO: 1 or 3 under high, moderate, or low stringency conditions. After amplification or isolation of a segment of a putative ortholog, that segment may be cloned and sequenced by standard techniques and utilized as a probe to isolate a complete cDNA or genomic clone. Alternatively, it is possible to initiate an EST project to generate a database of sequence information for the plant species of interest. In another approach, antibodies that specifically bind known MTP polypeptides are used for ortholog isolation. Western blot analysis can determine that an MTP ortholog (i.e., an orthologous protein) is present in a crude extract of a particular plant species. When reactivity is observed, the sequence encoding the candidate ortholog may be isolated by screening expression libraries representing the particular plant species. Expression libraries can be constructed in a variety of commercially available vectors, including lambda gt11, as described in Sambrook, et al., 1989. Once the candidate ortholog(s) are identified by any of these means, candidate orthologous sequence are used as bait (the “query”) for the reverse BLAST against sequences from tomato or other species in which MTP nucleic acid and/or polypeptide sequences have been identified.

Antibodies.

The present invention further provides anti-MTP polypeptide antibodies. The antibodies may be polyclonal, monoclonal, humanized, bispecific or heteroconjugate antibodies.

Polyclonal antibodies can be produced in a mammal, for example, following one or more injections of an immunizing agent, and preferably, an adjuvant. Typically, the immunizing agent and/or adjuvant will be injected into the mammal by a series of subcutaneous or intraperitoneal injections. The immunizing agent may include an MTP polypeptide or a fusion protein thereof. It may be useful to conjugate the antigen to a protein known to be immunogenic in the mammal being immunized.

Alternatively, the anti-MTP polypeptide antibodies may be monoclonal antibodies. Monoclonal antibodies may be produced by hybridomas, wherein a mouse, hamster, or other appropriate host animal, is immunized with an immunizing agent to elicit lymphocytes that produce or are capable of producing antibodies that will specifically bind to the immunizing agent (Kohler and Milstein, Nature 256:495, 1975). Monoclonal antibodies may also be made by recombinant DNA methods, such as those described in U.S. Pat. No. 4,816,567.

In one exemplary approach, anti-MTP polyclonal antibodies are used for gene isolation. Western blot analysis may be conducted to determine that MTP or a related protein is present in a crude extract of a particular plant species. When reactivity is observed, genes encoding the related protein may be isolated by screening expression libraries representing the particular plant species. Expression libraries can be constructed in a variety of commercially available vectors, including lambda gt11, as described in Sambrook, et al., supra.

Transgenic Plants

The MTP nucleotide sequence, protein sequence and phenotype find utility in modulated expression of the MTP protein and the development of non-native phenotypes associated with such modulated expression. In particular, up-regulation of MTP77 and/or MTP96 is associated with increased anthocyanin content in plants characterized by features that distinguish from wild type plants, including modified leaf color, modified flower color and modified fruit color.

In one aspect, the modified leaf, flower and fruit color of plants having increased cyanin content finds utility in the development of improved ornamental plants, fruits and/or cut flowers. In another aspect, the increased anthocyanin content in plants finds utility in plant-derived food, food additives, nutrition supplements, and natural dyes.

An MTP gene may be used to generate transgenic plants that produce flavonoids including anthocyanins and isoflavones. For example, a plant may be transformed with an MTP77 transgene, an MTP96 transgene, or both an MTP77 and MTP96 transgenes. Such transgenic plants may further comprise an ANT1 transgene. When separation from other plant material is desired, flavonoids may be extracted by any method known in the art (Yang et al., J Chromatogr A (2001) 928(2):163-170; Di Mauro et al., J. Agric. Food Chem (2002) 50:5968-5974; Matsumoto et al., J. Agric. Food Chem (2001) 49:1541-1545). An extracted flavonoid may be substantially purified or may be used in an unprocessed or partially processed state.

In one preferred embodiment, the invention provides transgenic tomato that produces at least one anthocyanin selected from delphinidin 3-rutinoside-5-glucoside, delphinidin 3-(coumaroyl)rutinoside-5-glucoside, delphinidin 3-(caffeoyl)rutinoside-5-glucoside, petunidin 3-rutinoside-5-glucoside, petunidin 3-(coumaroyl)rutinoside-5-glucoside, petunidin 3-(caffeoyl)rutinoside-5-glucoside, malvidin3-rutinoside-5-glucoside, malvidin 3-(coumaroyl)rutinoside-5-glucoside, and malvidin 3-(caffeoyl)rutinoside-5-glucoside. In a further preferred embodiment, the anthocyanin is produced at a level that is at least 5-, 10-, 20-, 50-, or 100-fold that observed in the non-transgenic plant.

In another preferred embodiment, the invention provides transgenic tobacco that produces at least one anthocyanin selected from cyanidin-3-glucoside and cyanidin-3-rutinoside. In a further preferred embodiment, the anthocyanin is produced at a level that is at least 5-, 10-, 20-, 50-, or 100-fold that observed in the non-transgenic plant.

Plants that over-expression an MTP gene in tomato can result in isoflavone production, which is otherwise undetectable. Accordingly, MTP genes can be used in the generation of transgenic soy or other legumes with altered isoflavone content or composition. MTP genes can also be used to produce isoflavones in plants other than legumes. In one embodiment, plants are generated that have increased glycitein content. In another embodiment, the isoflavone is produced at a level of at least 1.00 mg/100 g. Thus, the MTP gene may be used to generate transgenic plants that produce desired metabolites, including isoflavones. The isoflavones may be extracted by any method known in the art.

The methods described herein are generally applicable to all plants. In one aspect, the invention is directed to fruit- and vegetable-bearing plants. The invention is generally applicable to plants which produce fleshy fruits; for example but not limited to, tomato (Lycopersicum); grape (Vitas); ); strawberry (Fragaria); raspberry, blackberry, loganberry (Rubus); currants and gooseberry (Ribes); blueberry, bilberry, whortleberry, cranberry (Vaccinium); kiwifruit and Chinese gooseberry (Actinida); apple (Malus); pear (Pyrus); melons (Cucumis sp.) members of the Prunus genera, e.g. plum, cherry, nectarine and peach; sapota (Manilkara zapotilla); mango; avocado; apricot; peaches; cherries; pineapple; papaya; passion fruit; citrus; date palm; banana; plantain; and fig.

Similarly, the invention is applicable to vegetable plants, including, but not limited to sugar beets, green beans, broccoli, brussel sprouts, cabbage, celery, chard, cucumbers, eggplants, peppers, pumpkins, rhubarb, winter squash, summer squash, zucchini, lettuce, radish, carrot, pea, potato, corn, murraya and herbs.

In a related aspect, the invention is directed to the cut flower industry, grain-producing plants, oil-producing plants and nut-producing plants, as well as other crops including, but not limited to, cotton (Gossypium), alfalfa (Medicago sativa), flax (Linum usitatissimum), tobacco (Nicotiana), turfgrass (Poaceae family), and other forage crops. Suitable transformation techniques for these and other plants are known in the art.

A wide variety of transformation techniques exist in the art, and new techniques are continually becoming available. Any technique that is suitable for the target host plant can be employed within the scope of the present invention. For example, the constructs can be introduced in a variety of forms including, but not limited to as a strand of DNA, in a plasmid, or in an artificial chromosome. The introduction of the constructs into the target plant cells can be accomplished by a variety of techniques, including, but not limited to Agrobacterium-mediated transformation, electroporation, microinjection, microprojectile bombardment calcium-phosphate-DNA co-precipitation or liposome-mediated transformation of a heterologous nucleic acid construct comprising the MTP coding sequence. The transformation of the plant is preferably permanent, i.e. by integration of the introduced expression constructs into the host plant genome, so that the introduced constructs are passed onto successive plant generations.

In one embodiment, binary Ti-based vector systems may be used to transfer and confirm the association between enhanced expression of an identified gene with a particular plant trait or phenotype. Standard Agrobacterium binary vectors are known to those of skill in the art and many are commercially available, such as pBI121 (Clontech Laboratories, Palo Alto, Calif.).

The optimal procedure for transformation of plants with Agrobacterium vectors will vary with the type of plant being transformed. Exemplary methods for Agrobacterium-mediated transformation include transformation of explants of hypocotyl, shoot tip, stem or leaf tissue, derived from sterile seedlings and/or plantlets. Such transformed plants may be reproduced sexually, or by cell or tissue culture. Agrobacterium transformation has been previously described for a large number of different types of plants and methods for such transformation may be found in the scientific literature.

Depending upon the intended use, a heterologous nucleic acid construct may be made which comprises an MTP nucleic acid sequence, and which encodes the entire protein, or a biologically active portion thereof for transformation of plant cells and generation of transgenic plants.

The expression of an MTP nucleic acid sequence or an ortholog, homologue, variant or fragment thereof may be carried out under the control of a constitutive, inducible or regulatable promoter. In some cases expression of the MTP nucleic acid sequence or homologue, variant or fragment thereof may regulated in a developmental stage or tissue-associated or tissue-specific manner. Accordingly, expression of the nucleic acid coding sequences described herein may be regulated with respect to the level of expression, the tissue type(s) where expression takes place and/or developmental stage of expression leading to a wide spectrum of applications wherein the expression of an MTP coding sequence is modulated in a plant.

Strong promoters with enhancers may result in a high level of expression. When a low level of basal activity is desired, a weak promoter may be a better choice. Expression of MTP nucleic acid sequence or homologue, variant or fragment thereof may also be controlled at the level of transcription, by the use of cell type specific promoters or promoter elements in the plant expression vector.

Numerous promoters useful for heterologous gene expression are available. Exemplary constitutive promoters include the raspberry E4 promoter (U.S. Pat. Nos. 5,783,393 and 5,783,394), the 35S CaMV (Jones J D et al, Transgenic Res 1:285-297 1992), the CsVMV promoter (Verdaguer B et al., Plant Mol Biol 37:1055-1067, 1998) and the melon actin promoter. Exemplary tissue-specific promoters include the tomato E4 and E8 promoters (U.S. Pat. No. 5,859,330) and the tomato 2AII gene promoter (Van Haaren M J J et al., Plant Mol Bio 21:625-640, 1993).

When MTP sequences are intended for use as probes, a particular portion of an MTP encoding sequence, for example a highly conserved portion of a coding sequence may be used.

In yet another aspect, in some cases it may be desirable to inhibit the expression of endogenous MTP sequences in a host cell. Exemplary methods for practicing this aspect of the invention include, but are not limited to antisense suppression (Smith, et al., Nature 334:724-726, 1988); co-suppression (Napoli, et al, Plant Cell 2:279-289, 1990); ribozymes (PCT Publication WO 97/10328); and combinations of sense and antisense (Waterhouse, et al., Proc. Natl. Acad. Sci. USA 95:13959-13964, 1998). Methods for the suppression of endogenous sequences in a host cell typically employ the transcription or transcription and translation of at least a portion of the sequence to be suppressed. Such sequences may be homologous to coding as well as non-coding regions of the endogenous sequence. In some cases, it may be desirable to inhibit expression of the MTP nucleotide sequence. This may be accomplished using procedures generally employed by those of skill in the art together with the MTP nucleotide sequence provided herein.

Standard molecular and genetic tests may be performed to analyze the association between a cloned gene and an observed phenotype. A number of other techniques that are useful for determining (predicting or confirming) the function of a gene or gene product in plants are described below.

Generation of Mutated Plants with an MTP Phenotype

The invention further provides a method of identifying plants that have mutations in, or an allele of, endogenous MTP that confer an MTP phenotype, and generating progeny of these plants that also have the MTP phenotype and are not genetically modified. In one method, called “TILLING” (for Targeting Induced Local Lesions IN Genomes), mutations are induced in the seed of a plant of interest, for example, using EMS treatment. The resulting plants are grown and self-fertilized, and the progeny are used to prepare DNA samples. MTP-specific PCR is used to identify whether a mutated plant has an MTP mutation. Plants having MTP mutations may then be tested for the MTP phenotype, or alternatively, plants may be tested for the MTP phenotype, and then MTP-specific PCR is used to determine whether a plant having the MTP phenotype has a mutated MTP gene. TILLING can identify mutations that may alter the expression of specific genes or the activity of proteins encoded by these genes (see Colbert et al (2001) Plant Physiol 126:480-484; McCallum et al (2000) Nature Biotechnology 18:455-457).

In another method, a candidate gene/Quantitative Trait Locus (QTLs) approach can be used in a marker-assisted breeding program to identify alleles of or mutations in the MTP gene or orthologs of MTP that may confer the MTP phenotype (see Foolad et al., Theor Appl Genet. (2002) 104(6-7):945-958; Rothan et al., Theor Appl Genet (2002) 105(1):145-159); Dekkers and Hospital, Nat Rev Genet. (2002) January;3(1):22-32).

Thus, in a further aspect of the invention, an MTP nucleic acid is used to identify whether a plant having an MTP phenotype has a mutation in endogenous MTP or has a particular allele that causes the MTP phenotype compared to plants lacking the mutation or allele, and generating progeny of the identified plant that have inherited the MTP mutation or allele and have the MTP phenotype. The MTP plants generated can be used as non-genetically modified foods having increased flavonoid content, and can also be used for the same purposes described herein for transgenic MTP plants (e.g. extraction of natural dyes, etc.).

### EXAMPLES

Identification and Characterization of MTPs

In this study, we describe the phenotypic and molecular characterization of an activation-tagged tomato mutant in which a highly pigmented phenotype is directly associated with the overexpression of the tomato myb factor ANT1 (WO 02/055658).

Overexpression of ANT1 leads to the accumulation of anthocyanins in the leaves of transgenic plants, and also regulates downstream steps leading to the synthesis and accumulation of anthocyanins. ANT1 regulates genes encoding enzymes of both early and late steps of anthocyanin biosynthesis. In addition, ANT1 regulates genes encoding novel proteins in tomato that likely play a role in the synthesis and modification of anthocyanins as well as their transport and sequestration into the vacuole.

Plant Material:

Untransformed microtom (WT) was compared with the transgenic microtom overexpressing the ANT1 gene via a strong constitutive promoter. T2 seeds were surface sterilized and grown on TSG medium in the Conviron for three weeks. Only transgenic plants showing the pigmented phenotype were analyzed. At least 6 plants per sample were pooled prior to RNA extraction.

Northern Blot Hybridizations:

Total RNA was extracted from 3-week old seedlings using TriReagent according to the supplied protocol (Sigma). For each sample, 20 ug of total RNA was separated in a 1.2% agarose formaldehyde gel and transferred to Nytran Plus membrane (Schleicher and Schuell, Keene, N.H.) as described in Sambrook et al. (1989). Equal RNA loading was confirmed by methylene blue staining of the RNA on the membrane.

DNA fragments used as probes were PCR amplified from tomato SMART cDNA using oligonucleotide primers designed to dihydroflavonol reductase (DFR).

The probes used to validate the differentially expressed transcript were PCR amplified from the pCR2.1 vector with oligonucleotide primers complimentary to the vector flanking the TA cloning site. Amplification of all probe fragments was performed for 30 cycles in a Perkin Elmer 480 thermal cycler using a 60° C. annealing temperature and a 1 min extension.

The amplified probe fragments (about 50 ng) were labeled with [³²P]dCTP (NEN, Boston, Mass.) using the Ready-To-Go DNA labeling kit (Amersham Pharmacia, NJ). Hybridization conditions were as described by Church and Gilbert (1984). High stringency washes were performed under the following conditions: 65° C. in 1% SDS, 40 mM Sodium phosphate buffer pH 7, 1 mM EDTA. Northern blot hybridization signal was quantified with a PhosphorImager (Molecular Dynamics).

Suppression Subtractive Hybridization (SSH):

Two micrograms of total RNA were used to synthesize SMART cDNA according to the protocol supplied with CLONTECH's Super SMART PCR cDNA Synthesis Kit (K1054-1). Triplicate 100 ul amplification reactions were set up for each of the cDNAs; 17 cycles were determined to be optimal for amplification of the double-stranded cDNA. After amplification, the triplicate reactions were pooled, phenol chloroform extracted and ethanol-precipitated. The precipitated double-stranded SMART cDNA was resuspended in TE.

SMART cDNA was RsaI-digested and adaptors were ligated according the protocol supplied with the CLONTECH PCR-Select cDNA Subtraction Kit (K1804), Two rounds of subtractive hybridization were performed with the WT and ANT1 transgenic SMART cDNA samples, including both forward (MTP) and reverse subtractions (MTC). Primary and secondary PCR amplifications were performed using 27 cycles and 12 cycles, respectively. The resulting pools of differentially expressed fragments were each cloned into a TA cloning vector (pCR2.1, Stratagene). The ligation reactions were purified over a G-50 column prior to transformation into invaF′ competent cells (Invitrogen). Each transformation (MTC & MTP) was plated on selective media (ampicillin).

In order to estimate relative transcript abundance in each pool of cloned fragments, 48 colonies per transformation were picked for PCR colony screening with primers flanking the TA cloning site (M13 F&R as described above). The 96 reaction products (amplified insert) were separated on duplicate agarose gels, transferred to nylon membranes with 0.4M NaOH, and probed separately with SMART cDNA from either WT or ANT1 transgenic microtom. Probe labeling, hybridization and signal detection were performed as previously described. The average signal intensity of the 48 cloned fragments from the forward subtraction (MTP) was 2-fold greater than the average signal intensity of the reverse subtraction (MTC), indicating that the MTP pool indeed was enriched in up-regulated transcript fragments (data not shown).

The clones showing the highest fold change in expression between the WT and transgenic samples were selected for validation by Southern hybridization and for DNA sequencing. Plasmid DNA templates were sequenced using the M13F & R primers on an ABI3100 DNA sequencer. Vector, primer and poly(A) sequences were removed from the output prior to BLASTN analysis against the tomato EST collection in GenBank, assembled into the least number of contigs.

For the Southern hybridizations, SMART cDNA (3 ug/lane) was separated, transferred to nylon membrane (0.4M NaOH) and hybridized with labeled PCR fragments corresponding to candidate regulated transcript fragments. Hybridization with probes to ANT1, DFR and GST verify that these genes are upregulated in the ANT1 transgenic plants. The results also confirm that the SMART Southern results are similar to results from a northern blot hybridization, even including the ability to resolve different splice and polyadenylated forms of the GST & DPR transcripts.

The 5′ and 3′ ends of the MTP77 cDNA were amplified from SMART cDNA using nested sequence-specific primers and primers complementary to the adaptors on the ends of the SMART cDNA fragments. A full-length MTP77 cDNA clone was then amplified from SMART cDNA using sequence-specific primers designed based on the sequences of the 3′ and 5′ ends. The 1.7 kb fragment was cloned and sequenced.

Results & Discussion

The expression level of the ANT1 transgene corresponds to the intensity of the pigmented phenotype. The expression level of the ANT1 transgene also correlates with the expression level of a downstream gene encoding GST.

Validation of gene expression via SMART cDNA Southern hybridization is similar to northern blot hybridization and can resolve the presence of different splice forms of differentially expressed transcripts. DFR, a single copy gene in tomato, is represented by two trancript sizes resulting from alternate polyadenylation signals (Bongue-Bartelsman et al., 1994 Gene 138: 153-7.). The SMART cDNA Southern blot is able to detect these two cDNA sizes, which differ by approximately 100 bp. In addition, the SMART cDNA Southerns corroborate northern blot analysis of the GST transcript, shown to be present in two forms, corresponding to the spliced and unspliced forms. Approximately 40% of the total transcript is unspliced in the ANT1 transgenic, independent of the GST expression level. GST is represented as both spliced & unspliced forms, a possible point of regulation in the pathway. Splicing regulation has not been previously reported for this pathway, and may be a result of the high level of expression of the transgene controlling the phenotype in the transgenic tomato. Unspliced GST transcript was not detected in the pap1-D Arabidopsis mutant, for example (Borevitz et al. 2000 Plant Cell 12: 2383-2393.).

ANT1 Regulates a Variety of Genes Involved in Anthocyanin Accumulation

The overexpression of ANT1 results in the overexpression of genes encoding proteins in the early and late biosynthetic steps of anthocyanin biosynthesis. In addition, ANT1 appears to control expression of genes encoding proteins involved in the decoration and transport of anthocyanins into the vacuole. A summary of the validated differentially expressed transcripts in the ANT1 transgenic tomato is presented in Table 1. Up-regulation of all of the genes in Table 1 was confirmed by SMART cDNA southern analysis. In all cases, the up-regulated genes are undetectable in the leaves of WT tomato.

TABLE 1
Genes that are up-regulated in ANT1 transgenic tomato. Suppression
subtractive hybridization (SSH) fragments were cloned and sequenced. The SSH fragment
sequence was compared to a database containing tomato ESTs assembled into the least
number of contigs (BLASTN). The EST contig with the highest match to the SSH
fragment was assigned a putative identity based on a BLASTX search against the non-
redundant protein database in GenBank.

| | SSH | EST | | | |
|---|---|---|---|---|---|
| SSH | Insert | Contig | | BLASTN | |
| Clone | Size | Size | | Score | |
| Name | (bp) | (bp) | ESTs in the contig | (P) | Putative Identity |
| MT13 | 1004 | 498 | BE462282 BE462229 | 1232 (4.9e−51) | Myb factor; similar to |
| | | | AW626121 (Contig is | | Petunia AN2 (=ANT1) |
| | | | Seq ID No: 11) | | |
| MT16 | 480 | 1410 | BM411645 (Seq ID | 2382 (2.0e−103) | chalcone synthase |
| | | | No: 12) | | |
| MT2 | 546 | 1667 | BE461567 BE460511 | 941 (2.0e−38) | 5-O-glucosyl- |
| | | | BE459234 BE436963 | | transferase |
| | | | BE436886 BE436794 | | |
| | | | BE436417 BE436385 | | |
| | | | BE436300 BE436296 | | |
| | | | BE436116 BE435816 | | |
| | | | BE435361 BE435208 | | |
| | | | BE435015 BE434398 | | |
| | | | BE434141 BE434084 | | |
| | | | BE433520 BE433099 | | |
| | | | BE432679 BE432054 | | |
| | | | BE431801 AW933199 | | |
| | | | AW932099 AW931633 | | |
| | | | AW931609 AW930557 | | |
| | | | AW651280 AW651250 | | |
| | | | AW650795 AW648641 | | |
| | | | AW442216 AW442098 | | |
| | | | AW216889 AW220654 | | |
| | | | AW220655 AW220656 | | |
| | | | AW220874 AW221860 | | |
| | | | AW222352 (contig is | | |
| | | | (Seq ID No: 9) | | |
| MT11 | 281 | 673 | BI209061 AI775693 | 1400 (9.3e−59) | 3-O-glucosyl- |
| | | | BG628982 BG631865 | | transferase |
| | | | BG630259 (Contig is Seq | | |
| | | | ID No: 10) | | |
| MTP4 | 400 | 521 | AI778224 (Seq ID No: 6) | 730 (2.2e−28) | type-I GST similar to |
| | | | | | Petunia AN9 |
| MTP96 | 970 | 831 | BQ505699 (Seq ID No: 8) | 1753 (1.1e−72) | similar to chalcone |
| | | | | | isomerase |
| MTP2 | 301 | 362 | AI896332 (Seq ID No: 5) | 1463 (2.5e−61) | HD-GL2 similar to |
| | | | | | ANL2 |
| MTP77 | 390 | 620 | BE354224 (Seq ID No: 7) | 816 (2.4e−32) | permease, similar to |
| | | | | | TT12 & family). |

The ANT1 transgene itself, Myb factor (Petunia AN2 orthologue), was isolated by SSH, validating the experimental approach. ANT1 overexpression regulates both early (CHS) and late (DFR) steps of the anthocyanin biosynthetic pathway in tomato. In addition, genes likely encoding the “decorating” enzymes 3-O-glucosyltransferase and 5-O-gucosyltransferase are also regulated by ANT1 as well as a type-I GST, a flavonoid-binding protein required for vacuolar transport (similar to Petunia AN9). Three novel genes were also validated as being substantially up-regulated in the ANT1 transgenic line: a gene similar to chalcone isomerase (CHI-like), a HD-GL2 gene similar to Arabidopsis HD-GL2-protein, and a putative permease similar to proteins with about 10 TM helices required for vacoular transport of proanthocyanidins (Arabidopsis TT12 & family).

Genes encoding the “decorating” enzymes 3-O-glucosyltransferase and 5-O-gucosyltransferase are also regulated by ANT1. Anthocyanins are frequently glycosylated, and glucosyltransferases filling this role have been identified. In both Petunia and tomato, UDP-glucose:flavonoid glucosyltransferases are responsible for the glucosylation of anthocyanidins and anthocyanins that stabilize the molecules and are up-regulated coordinately with other anthocyanin biosynthetic genes (Yamazaki et al., 2002 Plant Mol Biol. 48:401-11; Bovy et al., 2002 Plant Cell. 14:2509-26.).

A gene with strong similarity to the Petunia AN9 gene encoding GST, required for efficient vacuolar sequestration (Mueller et al., 2000 Plant Physiol 123: 1561-1570), was up-regulated in the MTP ANT1 transgenic line. Anthocyanins are cytotoxic and unstable in the neutral pH of the cytoplasm. Therefore, sequestration of anthocyanins into the acidic vacuole is an important component of the pathway leading to anthocyanin accumulation. The transport of anthocyanins into the vacuole was long believed to involve transport of an anthocyanin-glutathione conjugate by a GS-X pump (Marrs et al., 1995 Nature 375: 397-400). However, more recent studies dispute the formation of an anthocyanin-glutathione conjugate (Mueller et al., 2000 Plant Physiol 123: 1561-1570), and suggest, instead, that GST acts as an anthocyanin binding protein that may serve as a chaperone.

Three new players in tomato anthocyanin synthesis and accumulation were identified in ANT1 transgenic tomato by SSH. One gene regulated in the ANT1 transgenic, MIP2, may encode a protein with similarity to the homeodomain-GLABRA2 (HD-GL2) class of transcription factors. The MTP2 fragment and the corresponding EST contig only represent a partial coding region. A similar gene product from Arabidopsis, ANTHOCYANINLESS2, was shown to be required for the accumulation of anthocyanins in subepidermal cells of vegetative tissues, but had no effect on proanthocyanidin accumulation in the seed coat (Kubo et al., 1999 Plant Cell 11: 1217-1226). In the ANT1 transgenic tomato leaves, anthocyanins accumulated primarily in the epidermal cells, and so, while the tomato MTP2 cDNA and Arabidopsis ANL2 gene products might both function in regulating the tissue-specific accumulation of anthocyanins, their role may be confined to different cell-types.

The MTP96 transcript, up-regulated in the ANT1 transgenic line, encodes a protein with similarity to chalcone isomerase (CHI). The CHI-like gene product encoded by MTP96 is most similar to the Arabidopsis At3g63170 gene product and is only 17% identical (32% similar) to the Petunia CHI-A gene product (gi|7331150). Similarity also exists with the following amino acid sequences: citrus (Citrus sinensis, gi|4126399); rice (Oryza sativa, gi|20152984); alfalfa (Medicago sativa CHI1, gi|116134 & CHI2, gi|116135); and petunia (Petunia×hybrida CHI-B, gi|68483).

The MTP96 product lacks the conserved residues reported to be involved in (2S)-naringenin binding and substrate preference determination (Jez et al., 2000 Nat Struct Biol. 7:786-91), suggesting that the substrate for this enzyme may be modified.

There is no report of a CHI gene from tomato in the public databases, though a CHI cDNA clone was reported to have been isolated recently from tomato (Bovy et al., 2002 Plant Cell. 14:2509-26). However, the reported tomato CHI transcript is not regulated by the heterologous expression of maize transcription factors that regulate other enzymatic steps in the flavonoid pathway (Bovy et al., supra). We speculate that the recently reported CHI transcript and the MTP96-encoded CHI-like protein isolated in this study function in separate tissues or even steps of flavonoid biosynthesis, with the CHI-like transcript involved directly in anthocyanin biosynthesis and accumulation in the leaves of tomato.

Finally, the MTP77 clone encoding a putative anthocyanin permease was characterized. The complete MTP77 cDNA sequence was assembled, translated and compared to the TT12 gene product (gi|27151710) and two other related Arabidopsis gene products: the At4g00350 gene product (gi|18411304); and the At4g25640 gene product (gi|15235172). The MTP77 cDNA encodes a protein that is 36% identical (56% similar) to TT12, but even more like At4g00350 (53% identical & 68% similar) and At4g25640 (61% identical & 71% similar). TT12 resembles multidrug secondary transporters in the MATE family, and is likely to mediate the vacuolar sequestration of proanthocyanidins in the seed coat of Arabidopsis (Debeaujon et al., 2001 Plant Cell. April 2001;13(4):853-71.). The similarity of the MTP77 permease to TT12 and its coregulation with the ANT1 transcription factor suggest that the gene product functions as an anthocyanin vacuolar transporter in tomato leaves. The high degree of amino acid sequence similarity between the tomato MTP77 permease and the Arabidopsis At4g00350 and At4g25640 gene products further suggests that these Arabidopsis genes may play a role in anthocyanin sequestration in vegetative tissues.

Anthocyanins are cytotoxic and unstable in the neutral pH of the cytoplasm. Therefore sequestration of anthocyanins into the acidic vacuole is an important component of the pathway leading to anthocyanin accumulation. The transport of anthocyanins into the vacuole was long believed to involve transport of an anthocyanin-glutathione conjugate by a GS-X pump (Marrs et al., 1995 Nature 375: 397-400). However, more recent studies dispute the formation of an anthocyanin-glutathione conjugate (Mueller et al., 2000 Plant Physiol 123: 1561-1570), opening the possibility of other mechanisms of vacuolar transport. In maize, the transcription factors C1/R and P activate anthocyanin biosynthesis. Expression profiling of maize suspension cells overexpressing these transcription factors led to the identification of both known and novel genes associated with the pathway (Bruce et al., 2000 Plant Cell 12:65-80). Phenylalanine ammonia lyase and a putative hydroxylase were up-regulated in the transgenic maize cell lines, along with a variety of other genes including one similar to a multi drug resistance transporter that may be involved in anthocyanin sequestration.

Mutant analysis in Arabidopsis has also led to the identification of genes involved in the cell-type specific accumulation of anthocyanins (ANL2; Kubo et al., 1999 Plant Cell 11: 1217-1226) and the vacuolar accumulation of proanthocyanidins (TT12; Debeaujon et al., 2001, supra). While the role of genes encoding HD-GL2 and TT12-like transporter proteins in flavonoid accumulation has been shown in Arabidopsis, the evidence has been restricted to a role in the seed coat, and their regulation by a myb factor (ANT1) has not been previously reported. It has already been shown that anthocyanin accumulation is controlled differently in the seed coat and in vegetative tissues (Kubo et al., 1999, supra; Borevitz et al.2000, supra). TT12 is unlikely to play a role in the accumulation of anthocyanins in the leaves, but the other closely related Arabidopsis genes may. Likewise, we predict that the permease encoded by MTP77 functions as a major vacuolar transporter of anthocyanins in the leaves of tomato, and that a similar gene product is likely to be found in Petunia and maize.

1. An isolated polynucleotide comprising a nucleic acid sequence which encodes or is complementary to a sequence which encodes a MTP polypeptide having at least 80% sequence identity to the amino acid sequence presented as SEQ ID NO:2 or SEQ ID NO:4.

2. A plant transformation vector comprising the isolated polynucleotide of claim 1.

3. A transgenic plant cell comprising the vector of claim 2.

4. A method of modifying anthocyanin content in a plant comprising introducing into progenitor cells of the plant, a plant transformation vector according to claim 2 and growing the transformed progenitor cells to produce a transgenic plant wherein said polynucleotide sequence is expressed and said transgenic plant exhibits increased anthocyanin content relative to the same type of plant which has not been so transformed.

5. A transgenic plant comprising a plant transformation vector comprising a nucleotide sequence that encodes or is complementary to a sequence that encodes an MTP polypeptide, whereby the transgenic plant has increased anthocyanin content relative to control plants.

6. The transgenic plant of claim 5 wherein the nucleotide sequence encodes MTP77.

7. The transgenic plant of claim 5 wherein the nucleotide sequence encodes MTP96.

8. A method of producing anthocyanin comprising extracting anthocyanin from a transgenic plant of any one of claims 5-7.


SEQUENCE LISTING (12 sequences)

SEQ ID NO: 1 - DNA, 1675 bp, Lycopersicon esculentum
aattttaaagtgtgaagcagtgtttacttacttcacaatggagacaccgttgctgaatggctactccggctccggcgaacgcaacgaccttatcggggcggacggggactaccgtccggcgaaaagtacaaaggattggtgggcaattttctgtgtggagacattgaaactgtggagaattggaggtcctatagcattcaacataatctgccaatatggagtgaattctcttacaaatatatttgttggacatcttggaaatgttgagctctctgctatttctattgctcagactgttattagtactttttcttttggcttcatgatgggaatgggtagtgcattggagacactatgtggccaggcatatggagcagggcaagttcacatgttgggcgtttatatgcaacgatccatcataatcctgctagctacttgtgtctttctattgcccatttacttattcaccactccacttctcgtgttgctaggccaagaaactgcaatagcggacctctctggaagatataccatgttactcatcccacagttgttttcacttgctatcaattttcctacttccaaatttcttcaagctcagagtaaagtggatgttctcgctgggattggattcgcagctgtgctggtgcacgctttgttcctctggctctttatatatacacttgaatggggtactaatggtgctgctatagcctttgacctcacgaattggcttactgcgatggctcaattagcatacgttgttggatggtgtaaggatgggtggaaagggttgtcgtggtcagcgttcaatgagatttgggcattcgtgagactttctattgcttcagctgttatgttatgccttgagatttggtacatgatgagtattattctccttgttggccatcttaacaacgccgtcatagcagttggctccatttcaatttgtatgaacatcaatggatgggagagcatgcttttcattggaattaatgctgccataagcatccgagtctcaaatgaactggggcaaggacatccaagagctaccaaatactcggtctacatcacggtgtttcagtccctcctaatagggatactctgcatggtaattgtactagtagctagagatcatctggccattatcttttcaaacagcaaggagatgcaagaagctgttgctgatcttgcttaccttctagggattaccatggtccttaatagcgttcagccggttatatcaggggttgctgttggaggtggatggcaagctttagtagcttatatcaatcttggttgctattatgtttttggactccctcttggatatcttcttggttatgtagcaaaattaggaacaaagggactttggttagggatgatagcaggggcagctttgcagactcttctactcttgattatactgtataagactaactggaacaaggaggttaacgacacgacggagcgtatgcgcaagtggggaggtcaagattttgagacacagaaatctgcagatgggcagttaactattgagaatggtggtgttgcttaagtttatgattgattcattgaaataatgtattttttcttgtttatgtttgagtttgaaacttgtaaaatctgtttcttttgttatacactcagcaatagaaacgtgggttttccaatt

SEQ ID NO: 2 - protein, 506 aa, Lycopersicon esculentum
METPLLNGYSGSGERNDLIGADGDYRPAKSTKDWWAIFCVETLKLWRIGGPIAFNIICQYGVNSLTNIFVGHLGNVELSAISIAQTVISTFSFGFMMGMGSALETLCGQAYGAGQVHMLGVYMQRSIIILLATCVFLLPIYLFTTPLLVLLGQETAIADLSGRYTMLLIPQLFSLAINFPTSKFLQAQSKVDVLAGIGFAAVLVHALFLWLFIYTLEWGTNGAAIAFDLTNWLTAMAQLAYVVGWCKDGWKGLSWSAFNEIWAFVRLSIASAVMLCLEIWYMMSIILLVGHLNNAVIAVGSISICMNINGWESMLFIGINAAISIRVSNELGQGHPRATKYSVYITVFQSLLIGILCMVIVLVARDHLAIIFSNSKEMQEAVADLAYLLGITMVLNSVQPVISGVAVGGGWQALVAYINLGCYYVFGLPLGYLLGYVAKLGTKGLWLGMIAGAALQTLLLLIILYKTNWNKEVNDTTERMRKWGGQDFETQKSADGQLTIENGGVA

SEQ ID NO: 3 - DNA, 1067 bp, Lycopersicon esculentum
gaattcggcttagcgtggtcgcggccgaggtacgcggggacagcaaaacagaaacacaaattcatatatagctatcttcctctttgcctataaacaataatcagtgtaaaaatgccaaccaccatggaagatgtcactgccaagattgatgcagttgaaatcgactccaagagtgttgttgctctgaaggagaaggcgatcgaaggtgaagtaaagactacagaaacaacacaagagaagactaatgaagctaaaagtaatccggagaaagaatccaatggtgcaactttcaaagaagaggaagttccagttgaggttgaacctaaaactggagtctcttttcctgtcaaacttgaagatggaaagcagttgaaagctgtaggtttaaggaagaagagcatgcttggcatgggcatcaaaatctatggctttggaatatatgcagacaatgagaaattgaaagacctcatgcagtccaagattgcgaaagcaccagcaaaaccaacaaaggagatgtatcaaatggtgattgatagtgatttgggaatgatgatgcggttggtaattgttttctctaacctaaccatgaacatggtaagaaagaactttgacgaaggcctcggagctgccatcaagaagcttacaggtggaaagaatgaggagctcacaaagaagatcatgggagaagcatctgatgacataaagctgaattctggttctgtaattgagatttcaaggcttccaggatatgtccttcagaccaaagtaaagggtgagattgtgagcaaggtggaaagtgaactactctgcagggccttcatctacatgtatttaggtgatgatccatttgacaaagaagccaaggagaagttcgggacatccatgctctctctgttttaagtagtaggtttccagcatttactagtctaggttgttaaagaataagctgtggtgactttgaactgatgaataatagctcaataaaagcatggttgattttcaaaaaaaaaaaaaaaaaaaaaaaaaaaaaaagtacctgcccgggcggccgctcgaaagccgaattc

SEQ ID NO: 4 - protein, 250 aa, Lycopersicon esculentum
MPTTMEDVTAKIDAVEIDSKSVVALKEKAIEGEVKTTETTQEKTNEAKSNPEKESNGATFKEEEVPVEVEPKTGVSFPVKLEDGKQLKAVGLRKKSMLGMGIKIYGFGIYADNEKLKDLMQSKIAKAPAKPTKEMYQMVIDSDLGMMMRLVIVFSNLTMNMVRKNFDEGLGAAIKKLTGGKNEELTKKIMGEASDDIKLNSGSVIEISRLPGYVLQTKVKGEIVSKVESELLCRAFIYMYLGDDPFDKEA

SEQ ID NO: 5 - DNA, 365 bp, Lycopersicon esculentum
gaattcggcttccgagcggccgcccgggcaggtactgaaggtgctaacagctgggatggcgcaattcagctaatgtttgcagaggtgcagatgttaacgccagtagtggggacgagagaagtgtattttgtgagatattgcaaacaaatgagtgcagcacagtgggggattgtggatgtttctgtcgacaaagttgaagccagcattgacgcttctctccttaaatgcagaaaattaccctcaggatgcattctacaagaacaatccaatgcacattgcaaggtgacgtgggtggaacacttggagtgccaaaaaaacatagtggactctctgtacctcggccgcgaccacgctaagccgaattc

SEQ ID NO: 6 - DNA, 450 bp, Lycopersicon esculentum
agcggccgcccgggcaggtactaggacatcacaacaaaaaaaaaaaagaggaaaaatggtagtgaaagtgtatggttcagcaatggctgcatgtccacaaagggtcatggtttgtcttatagaattgggagtcgattatgaacttatacatgttgatcttgattctctccagcagaaaaaacctgatttcttgcttttacaggtaatattttagttgtcgcttttaacttatcaaaagaaaatcatatttactcataacattaattacttatgcttcgaatcgctttgttatatattcttgcagccatttggacaggttcctgtcattgaagagggcgatttcaggcttttcggtaactatatactatcattattaaagccaggcccattgaggcaaatcagcccactccactagaagtacctcggccgcgaccacgctaagccgaattc

SEQ ID NO: 7 - DNA, 448 bp, Lycopersicon esculentum
gaattcggcttagcgtggtcgcggccgaggtactttttcttttggcttcatgatgggaatgggtagtgcattggagacactatgtggccaggcatatggagcagggcaagttcacatgttgggcgtttatatgcaacgatccatcataatcctgctagctacttgtgtctttctattgcccatttacttattcaccactccacttctcgtgttgctaggccaagaaactgcaatagcggacctctctggaagatataccatgttactcatcccacagttgttttcacttgctatcaattttcctacttccaaatttcttcaagctcagagtaaagtggatgttctcgctgggattggattcgcagctgtgctggtgcacgctttgttcctctggctctttatatatacacttgaatggggtacctgcccgggcggccgctccgaattc

SEQ ID NO: 8 - DNA, 1067 bp, Lycopersicon esculentum; identical to SEQ ID NO: 3

SEQ ID NO: 9 - DNA, 632 bp, Lycopersicon esculentum
gaattcggcttagcgtggtcgcggccgaggtacctgtcgtggcttttcctcaatggcccgatcaaatgacaaatgctaagcaaattgaagatgtgtggaagagtggagttagagtgaatgtgaatgaagatggtattgttgagagtgaagaattgaaaaggtgtattgaatgtgttatggatggaggggtaaaaggggaagaaatgagaaaaaacgcgaaaaaatggaaagaattggctagagaagctgtgaaagaaggtggatcttcacacaagaatttaaaggcttttattgatgaagttgccaaaggttattgatatgtgactttgtatcatttgcttattatttgttaagaataagatgagatagcttctctgttgctcggacctttgaaaaacgttgacaactgcatgttggatccttcaaaagtagtgcattttttttggaagatacgacatgagtgagacaacataggaaagttttgagcaacataggatagttaagtgttctttttatttctatcttattattgtgccttcaacttttctaataaaatccaatttgaaagattcatgccaaaaaaaaaaaaaaaaaaaaaaaaaagtacctgcccgggcggccgctcgaaattc

SEQ ID NO: 10 - DNA, 369 bp, Lycopersicon esculentum
gaattcggcttagcgtggtcgcggccgaggtacttttttcaatgaaggaaaagggaaagtattaaggaaaaatgttgaagggctaaaagaaaaagcattagaggctgtgaaattagataatgggagttcaatagaaaatttcaaggttctagttgagctaattaaatgtcacaagcctacttaatttgttccaaagtatgttaatcattgcaattaggtgatttactaaaaatgttcatctagaactttccctcgagcttcccgttgtaatcctttttcagtttcaatttatttgatctgttttcttattagaaaaaaaaaaaaaaaaaaaaaagtacctgcccgggcggccgctcgaaagccgaattc

SEQ ID NO: 11 - DNA, 1037 bp, Lycopersicon esculentum
  misc_feature (712)..(714): n is a, c, g, or t
gaattcggctttcgagcggccgcccgggcaggtacgcggggaagaaaaaatttggtgtaagctattttctttgaagtactgaggatacaacttcagagaaatttgtaagtttgaattcctgcagcccgggatgaacagtacatctatgtcttcattgggagtgagaaaaggttcatggactgatgaagaagattttcttctaagaaaatgtattgataagtatggtgaaggaaaatggcatcttgttcccataagagctggtctgaatagatgtcggaaaagttgtagattgaggtggctgaattatctaaggccacatatcaagagaggtgactttgaacaagatgaagtggatctcattttgaggcttcataagctcttaggcaacagatggtcacttattgctggtagacttcccggaaggacagctaacgatgtgaaaaactattggaacactaatcttctaaggaagttaaatactactaaaattgttcctcgcgaaaagattaacaataagtgtggagaaattagtactaagattgaaattataaaacctcaacgacgcaagtatttctcaagcacaatgaagaatgttacaaacaataatgtaattttggacgaggaggaacattgcgaggagataataagtgagaaacaactccagatgcatctatggacaacggagatacatggtggataaatttactggaannnatgcatgacgatattgagaagaatgagaggtgtattantatganaaacactacagtttgtactggagatatcacccattaatatggntgagtactcntgcacaggacatagcntgaaattgggtgatttccttattacacccccacaggagaaaagagattttgctgatgccttgattactgttaaaattaacgggcgggccattccgcgtcaaatggaaagtctaatgactgtccgcgcggtttaatttggtgtacggataagagggctctgagttaaaaaaaaaaccgcgacatgatctggcggtagatctgttatggttt

SEQ ID NO: 12 - DNA, 544 bp, Lycopersicon esculentum
gaattcggcttagcgtggtcgcggccgaggtacttctcttgtagccctaagtttctcaggctttaggcccaacttcaattcaacttggtccaaaatcgcaggcccgccagggtgagcgatccaaaatagagagttccagtcagatatacctagaggttgaaatgcttctagaaggctcttttcgatgtttttcgagataagcccaggaacatccttgagtaagtggaatgtaagcccaacctcacgtaggtgtccgtcaatagcgccttcgctatcggggacaagagtttgggctgctgagacgagttcaaataaaggtctttcgacccctataattgggtccgaacctataataattgcggccgccccatcaccaaaaagggcttggcctaccaagccatctaagtgagattcactgggtccacggaacgtaactgcagtgatctcagagcaaacaacaaggactctagcacccttgttgttctcagccaagtccttggctagccgaagtacctgcccgggcggccgctcgaaagccgaattc